(19) United States
(12) Patent Application Publication
(10) Pub. No.: US 2006/0110343 A1
(43) Pub. Date: May 25, 2006

(54) Cosmetic or dermopharmaceutical compositions comprising tyramine derivatives, method for preparing same, and use thereof

(75) Inventor: Karl Lintner, Rambouillet (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SEDERMA SAS, Le PERRAY-EN-YVELINES cedex (FR)

(21) Appl. No.: 10/519,118

(22) PCT Filed: Jun. 25, 2003
(86) PCT No.: PCT/FR03/01950

§ 371 Date: Sep. 29, 2005

(30) Foreign Application Priority Data
Jun. 26, 2002 (FR) 02/07965

(51) Int. Cl. A61K 8/42; A61K 8/46

(52) U.S. Cl. 424/62; 564/47; 564/86; 562/450; 977/926

(57) ABSTRACT

The invention concerns cosmetic or dermopharmaceutical compositions comprising tyramine derivatives and their salts. The invention also concerns the method for preparing same and use thereof for reducing pigmaentation.

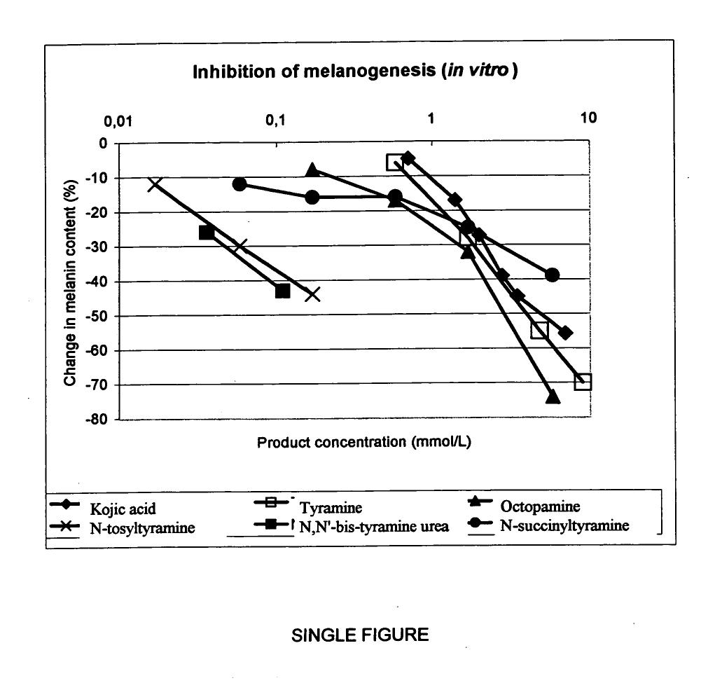

The present invention concerns cosmetic compositions or dermopharmaceutical compositions including tyramine derivatives together with the process for preparation of those compositions and use of the compositions for skin care, in particular with the objective of reducing hyperpigmentation.

The natural pigmentation of the skin stems from a mechanism that has now been clearly described: the melanocytes present in the stratum basale epidermidis produce melanin pigments which are synthesized in the melanosomes. Melanin synthesis (melanogenesis) increases under the action of UV radiation. The physiological function of tanning which ensues thus aims to protect the skin against UV radiation.

Various dysfunctions in the melanin production mechanism (due to an excess of external aggressions, hormonal disturbances or aging) induce the emergence of brown spots, particularly in the form of ephelides (freckles), and solar or senile lentigines.

Modifying the natural pigmentation of the skin is a desire shared by European, Asian and American women, although the underlying rationales differ: a white complexion is considered beautiful by some, while others seek to attenuate senile lentigines, considered to reveal aging. In Asia, as is the case in Europe and America, controlling skin pigmentation is thus a sensitive subject and the object of considerable demand.

Three key enzymes are involved in melanogenesis: tyrosinase and tyrosine-related proteins (TRP-1 and TRP-2). All three are glycoproteins located in the melanosome membrane. Out of the three, tyrosinase is the limiting enzyme in that it catalyzes the first two stages in pigment formation: ortho-hydroxylation of tyrosine to yield L-DOPA, then oxidation of the latter to yield dopaquinone. TRP-1 and TRP-2 are reported to intervene, in part, by stabilizing tyrosine hydroxylase.

In addition, it is known that stimulation of melanogenesis involves increasing intracellular cAMP levels. cAMP regulates the action of a protein kinase, PKC-b, whose ability to phosphorylate tyrosinase is determinant in melanin synthesis. In support of this mechanism, it will be observed that UV radiation very significantly increases PKC-b in cultured melanocytes.

Lastly, the role played by intracellular calcium in melanocyte metabolism is also undoubtedly to be taken into account.

To influence skin pigmentation, it is therefore possible to envisage degrading melanin, offering melanogenesis inhibitors which interact with the various targets described above, or even inhibiting the distribution of melanin in the epidermal cell layers.

However, the most frequently selected target is undoubtedly tyrosine hydroxylase, since it constitutes a limiting step in the process.

For a considerable time, depigmentation or lightening the skin was achieved using very potent products such as hydroquinone, sulfur- or non-sulfur-containing phenolic compounds and ascorbic acid. However, those products were not devoid of irreversible hypopigmentation effects and induced irritation. All those products are to be used in an efficacy/safety context that is not appropriate for cosmetics.

In the cosmetic field, the problem was tackled by using various retinoid derivatives, AHA, kojic acid and arbutin.

Hydroquinone, arbutin and kojic acid were developed for their competitive inhibition of tyrosinase or inhibition of the catalytic activity indispensable to tyrosinase function by chelation of copper ions. However, those products are difficult to use and may induce adverse effects.

There is thus a strong demand for innovative cosmetic products that are effective in vivo and non-toxic.

We discovered, quite surprisingly, that certain tyramine derivatives are endowed with a strong inhibitory potential with respect to melanogenesis that is greater than that of tyramine itself. This was described in patent application FR-A-2 813 188.

The invention constituting the subject of the present application resides in the fact that we have discovered and demonstrated that tyramine derivatives of general formula I reduce melanin production in an effective and non-toxic manner. The tyramine derivatives that constitute the subject of the present patent application are also of value in that they have superior bioavailability, solubility, activity, stability or toxicological profile.

The present invention thus addresses cosmetic or dermopharmaceutical compositions containing an excipient that is acceptable in cosmetic terms and, alone or in combination, a compound with the following general formula I: 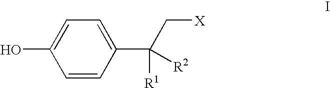
in which:

the group X is a —NR³R⁴ or —N═CR⁵R⁶ group,

each of the groups R¹ and R², which may be the same or different, consists in a hydrogen or halogen atom or an alkyl, aryl, aralkyl, acyl, alcohol or alkoxy group,

each of the groups R³ and R⁴, which may be the same or different, consists in a hydrogen atom or an alkyl, aryl, aralkyl, acyl, sulfonyl or sugar group,

each of the groups R⁵ and R⁶, which may be the same or different, consists in a hydrogen atom or alkyl, aryl or aralkyl group.

Compounds of general formula I may exist in free form or in the form of a salt formed with an acid that is acceptable in cosmetic terms. The present invention includes both the free forms and the salts of those compounds.

The present invention does not concern cosmetic or dermopharmaceutical compositions containing tyramine (formula I, X=—NR³R⁴, R¹R═R³═R⁴═H) or its derivatives formed by bonding, on the OH or NH₂ group, any linear or branched, saturated or unsaturated, alkyl chain, which may be hydroxylated or contain sulfur or not contain sulfur, consisting of 1 to 24 carbon atoms. The present invention also does not concern compositions containing synephrine (formula I, X=—NR³R⁴, R═OH, R²═R³═H, R⁴═CH₃).

In the context of the present invention, the term ‘acid acceptable in cosmetic terms’ is taken to mean any non-toxic acid, including organic and inorganic acids. Such acids include acetic, para-aminobenzoic, ascorbic, aspartic, benzenesulfonic, benzoic, bismethylene salicylic, hydrobromic, hydrochloric, cinnamic, citraconic, citric, ethanedisulfonic, fumaric, gluconic, glutamic, glyconic, itaconic, lactic, maleic, malic, mandelic, methanesulfonic, mucic, nitric, oxalic, palmitic, pamoic, pantothenic, phosphoric, propionic, salicylic, stearic, succinic, sulfamic, sulfuric, tartaric and para-toluenesulfonic acid. Hydrochloric acid and acetic acid are particularly preferred.

In the context of the present invention, the term ‘alkyl group’ is taken to mean any alkyl group of 1 to 20 carbon atoms, linear or branched, substituted or not substituted (substituted, in particular, by an alcohol, carboxylic acid or amine) and saturated or unsaturated. In particular, an alkyl group may be the methyl group. arabinose, fructose, galactose, mannose, maltose, lactose, sucrose or cellobiose groups.

The said constituents of formula I may contain a center of asymmetry and thus exist in the form of optical isomers. The present invention covers each of the optical isomers separately and any mixture of those isomers.

Under the terms of the invention, the cosmetic and dermopharmaceutical compositions that are particularly advantageous are those containing octopamine (formula I, X=—NR₃R₄, R¹═OH, R²═R³═R⁴═H), N-succinyltyramine (formula II), N-tosyltyramine (formula III) or N,N′-bis-tyramine urea (formula IV).

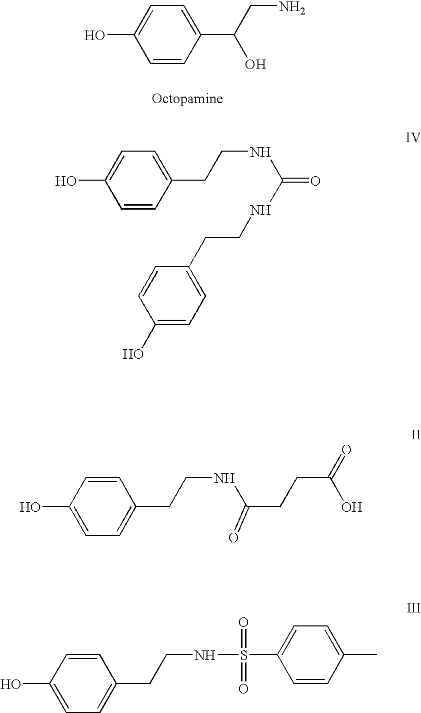

The present invention also covers the method of preparation of the compounds of general formula I. The preparation of those compounds consists, in particular, in reacting tyramine or octopamine or one of their salts, in the presence or absence of a base, with a carbonyl or sulfonyl derivative, in the presence or absence of a coupling reagent used in peptide synthesis (in particular, carbodiimide, acylimidazole, chloroformate, BOP, CDI, DCC, EEDQ, HTBU, PyBOP®, PyBroP®, TBTU, WSC.HCl available, for example, from Novabiochem). The carbonyl or sulfonyl derivative may, in particular, be an aldehyde, an activated or inactivated ester, a carboxylic or sulfonic acid chloride, an anhydride or an isocyanate.

Tyramine and octopamine, the starting compounds for the syntheses, are commercially available or may be prepared from starting materials commercially available using known processes.

The preferred compounds of formula I, II, III or IV are those obtained by the process described above from a tyramine salt.

The compounds of general formula I are used in cosmetic and dermopharmaceutical compositions as per the invention at concentrations which may range from 0.0001 (m/m) to 50% (m/m) but preferably between 0.001 (m/m) and 20% (m/m).

Said compounds may be used individually or in premixes in any galenic form, such as: lotions, milks or emollient creams; milks or creams for skin care or hair care; make-up-removing cleansing creams, lotions, or milks; foundation tint bases; sun-screen lotions, milks, or creams; artificial suntan lotions, milks, or creams; shaving creams and foams; aftershave lotions; shampoos, lipsticks, mascaras, or nail varnishes.

These compositions can also be presented in the form of lipsticks intended to apply colour or to protect the lips from cracking, or of make-up products for the eyes or tints and tint bases for the face.

When the compositions according to the invention are presented in the form of water-in-oil or oil-in-water emulsions, the fatty phase consists essentially of a mixture of fatty substances obtained by extraction or synthesis, with at least one oil and possibly another fatty substance. The fatty phase of the emulsions may constitute 5 to 60% of the total weight of the emulsion.

The aqueous phase of the said emulsions constitutes preferably 30 to 85% of the total weight of the emulsion. The proportion of the emulsifying agent may be between 1 and 20%, and preferably between 2 and 12% of the total emulsion weight. When the compositions according to the invention are presented in the form of oily, oleo-alcoholic, or aqueous-alcoholic lotions they may constitute, for example, sun-screen lotions containing a filter absorbing UV radiation or softening lotions for skin; the oily lotions may in addition constitute foam oils containing oil-soluble surfactant, bath oils, etc.

Among the principal adjuvants that may be present in compositions according to the invention one may cite organic or aqueous-glycolic solvents, including MP-diol and polyglycerols, fatty substances obtained by extraction or synthesis, ionic or non-ionic thickeners, softeners, opacifiers, stabilizers, emollients, silicones, α- or β-hydroxy acids, antifoaming agents, moisturizing agents, vitamins, perfumes, preservatives, sequestrating agents, colouring agents, gel-forming and viscosity-increasing polymers, surfactants and emulsifiers, other water- or fat-soluble active principles, plant extracts, tissue extracts, marine extracts, sun filters, and antioxidants.

The more particularly preferred mono- or poly-alcohols are chosen from among ethanol, isopropanol, propylene glycol, glycerol, and sorbitol.

As the fatty substance, among mineral oils one may cite liquid petrolatum; among animal oils whale oil, shark oil, seal oil, menhaden oil, halibut liver oil, cod liver oil, tunny-fish oil, turtle oil, neat's foot oil, horse foot oil, sheep's foot oil, mink oil, otter oil, marmot oil, etc.; and among vegetable oils almond oil, wheat germ oil, jojoba oil, sesame oil, sunflower seed oil, palm oil, walnut oil, shea nut oil, shorea oil, macadamia nut oil, blackcurrant seed oil, and the like.

Among the fatty acid esters one may use esters of C₁₂ to C₂₂ acids, saturated or unsaturated, and lower alcohols such as isopropanol or glycerol or aliphatic C₈ to C₂₂ alcohols, straight-chain or branched, saturated or unsaturated, or C₁₀-C₂₂ alkane 1,2-diols.

As the fatty substance one may also cite vaseline, paraffin, lanolin, hydrogenated lanolin, tallow, acetylated lanolin, and silicone oils.

Among waxes one may cite Sipol wax, lanolin wax, beeswax, Candelilla wax, monocrystalline wax, Carnauba wax, spermaceti, cocoa butter, karité nut butter, silicone waxes, hydrogenated oils solidified at 25° C., sucroglycerides, oleates, myristates, linoleates, and stearates of calcium, magnesium, and aluminium.

Among the aliphatic alcohols one may cite lauryl alcohol, cetyl alcohol, myristyl alcohol, stearyl alcohol, palmityl alcohol, oleyl alcohol, and Guerbet's alcohols such as 2-decyltetradecanol or 2-hexyldecanol. As emulsifying agents among the aliphatic polyoxyethylenated alcohols one may cite lauryl, cetyl, stearyl, and oleyl alcohols containing 2 to 20 moles of ethylene oxide, and among the glycerol alkoyl ethers C₁₂-C₁₈ alcohols containing 2-10 moles of glycerol. It may also be useful to include thickeners such as cellulose derivatives, polyacrylic acid derivatives, guar gum, carouba gum, or xanthan gum.

The composition according to the invention can also contain adjuvants commonly used in cosmetics and in dermatology, and in particular moisturizing agents, softeners, products for the treatment of skin conditions, sun filters, germicides, colouring agents, preservatives, perfumes, and propellants.

When the compositions according to the invention are in the form of dispersions, these may be dispersions of lecithin in water in the presence of a surfactant or they may be aqueous dispersions of lipid spherules consisting of organized molecular layers enclosing an encapsulated aqueous phase. The lipid compounds may be long-chain alcohols and diols, sterols such as cholesterol, phospholipids, cholesteryl sulfate and phosphate, long-chain amines and their quaternary ammonium derivatives, dihydroxyalkylamines, polyoxyethylenated aliphatic amines, long-chain amino alcohol esters, their salts and quaternary ammonium derivatives, phosphate esters of aliphatic alcohols such as hydrogen dicetyl phosphate or its sodium salt, alkyl sulfates such as sodium cetyl sulfate, fatty acids in the form of salts, or else lipids of the type of those described in French Patents Nos. 2 315 991, 1 477 048, and 2 091 516 or in international patent applications WO 83/01571 and WO 92/08685.

As other lipids one may use, for example, lipids containing a lipophilic long chain of 12 to 30 carbon atoms, saturated or unsaturated, branched or straight-chain, for example an oleyl, lanolyl, tetradecyl, hexadecyl, isostearyl, lauryl, or alkoylphenyl chain. The hydrophilic group in these lipids may be ionic or non-ionic. The non-ionic groups may be groups derived from polyethylene glycol. One can also use advantageously, as lipids forming the lamellar phase, polyglycol ethers such as those described in French Patents Nos. 1 477 048, 2 091 516, 2 465 780, and 2 482 128.

The ionic group may advantageously be a group derived from an amphoteric, anionic, or cationic compound.

Some other lipids described in international patent application WO 83/01571 as suitable for the formation of vesicles are glycolipids such as lactosylceramide, galactocerebroside, gangliosides and trihexosylceramide, as well as phospholipids such as phosphatidylglycerol and phosphatidylinositol.

The active substances may be substances of nutritional or pharmaceutical interest or ones having a cosmetic activity. When they are water-soluble they may be dissolved to produce a homogeneous solution or they are in the aqueous phase encapsulated within the vesicles. The water-soluble substances having a cosmetic and/or pharmaceutical activity may be products intended for skin and hair care or treatment, such as for example moisturizers such as glycerol, sorbitol, pentaerythritol, pyrrolidine acid and its salts; artificial suntan agents such as dihydroxyacetone, erythrulose, glyceraldehyde, γ-dialdehydes such as tartaric aldehyde, these products being possibly associated with colouring agents; water-soluble sun filters; antiperspirants, deodorants, astringents, fresheners, tonics, healing products, keratolytics, depilatories, scents; plant tissue extracts such as polysaccharides; water-soluble colorants; anti-dandruff agents; antiseborrheic agents, oxidants such as bleaching agents, for example hydrogen peroxide; and reducing agents such as thioglycolic acid and its salts.

Mention can also be made of vitamins, hormones, enzymes such as superoxide dismutase, vaccines, antiinflammatories such as hydrocortisone, antibiotics, bactericidal agents, cytotoxic agents, or antitumour agents.

When the active substances are oil-soluble they may be incorporated in the walls of the vesicles. They may be chosen from the group formed by oil-soluble sun filters, substances intended for improving of the condition of dry or old skin, tocopherols, vitamins E, F, or A or their esters, retinoic acid, antioxidants, essential fatty acids, glycyrrhetinic acid, keratolytics, and carotenoids.

Compounds of general formula I can be used in cosmetic compositions in accordance with the invention either as individual additions or as a premix in a suitable excipient, and be in the form of solution, dispersion, emulsion, paste, or powder. They may be included individually or together in vehicles consisting of cosmetic carriers such as macro-, micro-, or nanocapsules, liposomes or chylomicrons, macro-, micro-, or nanoparticles or microsponges. They may also be adsorbed on organic polymer powders, talcs, bentonites, or other inorganic supports.

They may be used in any form whatsoever, or in a form bound to or incorporated in or absorbed in or adsorbed on macro-, micro-, and nanoparticles, or macro-, micro-, and nanocapsules, for the treatment of textiles, natural or synthetic fibres, wools, and any materials that may be used for clothing or for day or night underwear intended to come into contact with the skin, such as tights, underclothes, handkerchiefs, or cloths, to exert their cosmetic effect via this skin/textile contact and to permit continuous topical delivery.

The present invention also covers use of cosmetic or dermopharmaceutical compositions containing compounds of general formula I with the exception of tyramine and its derivatives formed by bonding, on the OH or NH₂ group, any linear or branched, saturated or unsaturated, alkyl chain, which may be hydroxylated or contain sulfur or not contain sulfur, consisting of 1 to 24 carbon atoms, and with the exception of synephrine (formula I, X=—NR³R⁴, R¹═OH, R²═R³═H, R⁴═CH₃) in order to reduce melanin production with the aim of decreasing pigmentation, in particular to lighten the complexion, attenuate senile lentigines, homogenize skin color, or lighten any pigmentation associated with melanin, including that of the hair.

The present invention also concerns use of compounds of formulae I, II, III or IV and octopamine, alone or incorporated in cosmetic or dermopharmaceutical compositions as per the invention for the preparation of medicinal products for inhibition of melanogenesis or intended for skin care, particularly lightening the skin and reducing its color on exposure to natural or artificial UV radiation.

Examples are given below as a non-restrictive illustration of implementation of the present invention.

### EXAMPLE NO. 1

### Synthesis of N-succinyl-tyramine (compound II)

To a solution of tyramine chlorhydrate de tyramine (1.00 g; 5.76 mmoles) in 20 ml of THF are added, at room temperature, 1 equivalent of potassium carbonate (K₂CO₃) (0.80 g; 5.76 mmoles) and 1.04 equivalent of succinic anhydride (0.60 g; 5.99 mmols). After one night of stirring at room temperature, the mixture is hydrolyzed by addition of water (10 ml) and washed by addition of 4 g of amberlite resin IR120 (R—SO₃H) and stirring 15 minutes (pH=0-1). After filtration and water washing, the THF is cold evaporated. 0.92 g (3.88 mmoles; 67.3%) of N-succinyl-tyramine are isolated as white crystals.

C₁₂H₁₅NO₄

MM=237.257 gmol⁻¹

Melting Point: 135-136° C.

CHN: Calculated: 60.75% C, 6.35% H, 5.90% N, Found: 61.33% C, 6.05% H, 5.86% N.

Infra Red: 3313; 3055; 2930; 1694; 1643; 1542; 1517; 1426; 1238; 1208 cm⁻¹

### EXAMPLE NO. 2

### Synthesis of N-tosyl-tyramine (compound III)

To a solution of tyramine chlorhydrate (0.20 g; 1.15 mmols) in 4 ml of THF are added, at room temperature, 1 equivalent of potassium carbonate (K₂CO₃) (0.16 g; 1.16 mmols) and 1.10 equivalent of tosyl chloride (0.12 g; 1.27 mmols). After one night of stirring at room temperature, the mixture is hydrolyzed by addition of water (4 ml) and washed by addition of 4 g of amberlite resin IR120 (R—SO₃H) and stirring 1 hour. After filtration and washing with water then THF, solvant is cold evaporated and the solid is filtrated. 0.19 g (0.65 mmols; 56.00%) of N-tosyl-tyramine are isolated as white crystals.

C₁₅H₁₇NSO₃

MM=291.37 gmol⁻¹

Melting Point: 169-172° C.

CHN: Calculated: 61.83% C, 5.88% H, 4.81% N, Found: 61.94%; C, 5.83% H, 4.78% N.

Infra Red: 3335; 3220; 1613; 1598; 1515; 1435; 1312; 1229; 1149; 1063; 913; 833; 812 cm⁻¹

### EXAMPLE NO. 3

### Synthesis of N,N′-bis-tyramine-urea (compound IV)

To a solution of tyramine chlorhydrate (0.20 g; 1.15 mmols) in 4 ml of THF are added, at room temperature, 1 equivalent of potassium carbonate (K₂CO₃) (0.16 g; 1.16 mmols) and 0.54 equivalent of carbonyl-diimidazole (0.10 g; 0.62 mmol). After two days of stirring at room temperature, the mixture is hydrolyzed by addition of water (4 ml) and THF (4 ml) and washed by addition of 4 g of amberlite resin IR120 (R—SO₃H) and stirring 15 minutes. After filtration and washing with 4 ml of THF then 4 ml of water, solvant is cold evaporated and the solid is filtrated. 0.09 g (0.27 mmols; 43.20%) of N,N′-bis-tyramine-urea are isolated as white crystals.

C₁₇H₂₀N₂O₃

MM=300.3605 gmol⁻¹

Melting Point: 95-98° C.

Mass Spectrometry: (m/z)=301.2 [M+H]⁺

CHN: Calculated: 67.98% C, 6.71% H, 9.33% N Found: 67.66% C, 6.73% H, 9.36% N.

Infra Red: 3336; 3104; 2930; 1605; 1515; 1445; 1240; 1169; 1050; 822 cm⁻¹

EXAMPLE NO. 4
Day cream

| | g/100 g |
|---|---|
| Volpo S20 | 2.4 |
| Volpo S2 | 2.6 |
| Prostearyl 15 | 8.0 |
| Beeswax | 0.5 |
| Stearoxy dimethicone | 3.0 |
| Propylene glycol | 3.0 |
| Carbomer | 0.25 |
| NaOH 30% | 0.25 |
| Octopamine | 0.1 |
| Water & preservatives | qs 100 g |

This emulsion is used to lighten and moisturize face skin.

EXAMPLE NO. 5
Moisturizing and lightening body milk

| | |
|---|---|
| Crillet 3 | 2.5 |
| Novol | 0.9 |
| Fluilan | 2.5 |
| Carbopol | 9400.3 |
| Beeswax | 2.0 |
| NaOH 30% | 0.1 |
| Glycerine | 5.0 |
| N-tosyl-tyramine (III) | 0.01 |
| Water & preservatives | qs 100 g |

EXAMPLE NO. 6
Lightening gel

| | g/100 g |
|---|---|
| Carbomer | 0.3 |
| Propylene glycol | 2.0 |
| Glycerine | 1.0 |
| White petrolatum | 1.5 |
| Cylomethicone | 6.0 |
| Crodacol C90 | 0.5 |
| Lubrajel MS | 10.0 |
| Triethanolamine | 0.3 |
| N-succinyl-tyramine (II) | 0.02 |
| Water, preservatives & perfume | qs 100 g |

EXAMPLE NO. 7
Whitening cream

| | g/100 g |
|---|---|
| Cromul EM 1207 | 2.4 |
| Volpo S | 22.6 |
| Prostearyl | 158.0 |
| Beeswax | 0.5 |
| Stearoxy dimethicone | 3.0 |
| Propylene glycol | 3.0 |
| Carbopol 941 | 0.25 |
| Triethanolamine | 0.25 |
| N,N′-bis-tyramine-urea (IV) | 0.007 |
| Water, preservatives & perfume | qs 100 g |

### EXAMPLE NO. 8

### Inhibition of Melanin Synthesis (In Vitro)

The efficacy of the products on melanization was tested on a cultured stable cell line.

Melanocytes of line B16 were used in the study. Line B16 is conventionally used to test variations in melanin levels. The cells are incubated in the presence of the test product for 48 hours while the control cells are incubated in the culture medium alone.

After 48 hours, the total melanin (phaeomelanin and eumelanin) present in the cells is determined after cell lysis and dissolution in sodium hydroxide. The assay is colorimetric.

The melanin level produced under exposure to the test product at various concentrations are compared to those obtained with the control cells. The data are normalized on the protein content of the sample.

The single FIGURE shows the variation in melanin synthesis under exposure to kojic acid for 48 hours (positive control), on the one hand, and under exposure to a number of tyramine derivatives, on the other hand. The inhibition of synthesis observed after 48 hours of exposure to those products is dependent on the test concentration. Inhibition varies from −5 to −70%. This demonstrates that all the compounds have inhibitory activities on melanogenesis that are of great interest and superior to those of tyramine itself, particularly at low concentrations (≦0.8 mmol/L)

The examples of the new compounds derived from tyramine and the cosmetic compositions containing them and their uses are not restrictive.

The cosmetic or dermopharmaceutical compositions may also be used in the preparation of medicinal products intended for skin care, particularly skin lightening and reducing its coloration under exposure to natural or artificial UV radiation. Moreover, the compounds and compositions that are the subject of the present patent may be used to manufacture cloth, textiles and clothing with a cosmetic effect, in particular for lightening the skin or hair.

1-17. (canceled)

18. A cosmetic or dermopharmaceutical composition comprising:
an excipient acceptable in cosmetic terms; and
a compound with the general formula I: 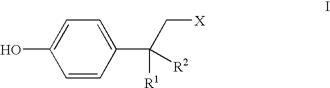
its salts with an acid acceptable in cosmetic terms, and its isomers, in which:
X is —NR³R⁴ or —N═CR⁵R⁶;
each of R¹ and R², which may be the same or different, is a hydrogen, a halogen, an alkyl, aryl, aralkyl, acyl, alcohol or alkoxy;
each of R³ and R⁴, which may be the same or different, is a hydrogen atom, alkyl, aryl, aralkyl, acyl, sulfonyl, or a sugar;
each of R⁵ and R⁶, which may be the same or different, is a hydrogen, alkyl, aryl, or aralkyl;
with the exception that formula I does not comprise tyramine and its derivatives formed by bonding on the OH or to X, when X is NH₂, any alkyl chain, comprising from 1 to 24 carbon atoms, and with the further exception that the formula I does not comprise synephrine.

19. The cosmetic or dermopharmaceutical composition of claim 18, wherein said compound is present at a concentration between 0.0001 (m/m) and 50% (m/m).

20. The cosmetic or dermatological composition of claim 18, wherein said compound is present at a concentration between 0.001 (m/m) and 20% (m/m).

21. The cosmetic or dermopharmaceutical composition of claim 18, wherein X is —NR³R⁴, R¹ is OH, R²═R³═R⁴ is H and said compound is octopamine or one of its salts.

22. The cosmetic or dermopharmaceutical composition of claim 18, wherein said compound comprises N-succinyl-tyramine with the following formula II: 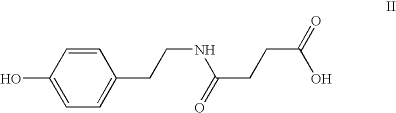
or one of its salts.

23. The cosmetic or dermopharmaceutical composition of claim 18, wherein said compound comprises N-tosyltyramine with the following formula III: 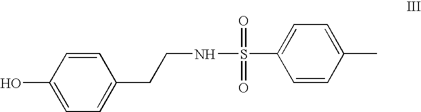
or one of its salts.

24. The cosmetic or dermopharmaceutical composition of claim 18, wherein said compound comprises N,N′-bistyramine urea with the following formula IV: 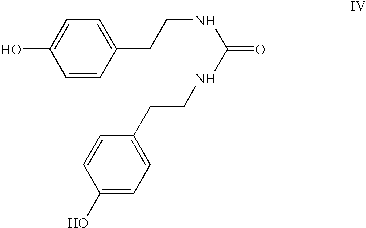
or one of its salts.

25. The cosmetic or dermopharmaceutical composition of claim 18, wherein said compound is in the form of a solution, dispersion, emulsion, paste, or powder.

26. The cosmetic or dermopharmaceutical composition of claim 18, wherein said compound may be part of, or contained within, a macrocapsule, microcapsule, nanocapsule, liposome, chylomicron, macroparticle, microparticle or nanoparticle, macrosponge, microsponge or nanosponge, or may be adsorbed on an organic polymer powder, talc, bentonite or inorganic support.

27. The cosmetic or dermopharmaceutical composition of claim 18, wherein said compound is in the form of a lotion, milk, emollient, or cream.

28. The cosmetic or dermopharmaceutical composition of claim 18, wherein said compound is a formulation for skin care or hair care, make-up-removing base, foundation tint base, sun-screen, artificial suntan base, shaving base, aftershave, shampoo, lipstick, mascara, or nail varnish.

29. The cosmetic or dermopharmaceutical composition of claim 18, further comprising at least one adjuvant, organic or hydroglycolic solvent, fatty substance obtained by extraction or synthesis, ionic or non-ionic thickener, softener, opacifier, stabilizer, emollient, silicone, α-hydroxy acid, antifoaming agent, moisturizing agent, vitamin, perfume, preservative, sequestrating agent, coloring agent, gel-forming and viscosity-increasing polymer, surfactant and emulsifier, other water- or fat-soluble active principle, plant extract, tissue extract, marine extract, sun filter, or antioxidant.

30. A process for preparing the compound of claim 18, said process comprising:
reacting tyramine, or one its salts, with a carbonyl or sulfonyl derivative.

31. The process of claim 30 comprising reacting tyramine, or one of its salts, in the presence of a base.

32. The process of claim 30 comprising reacting tyramine, or one of its salts, in the presence of a coupling reagent used in peptide synthesis.

33. A process for preparing the compound of claim 18, said process comprising:
reacting octopamine, or one of its salts, with a carbonyl or sulfonyl derivative.

34. The process of claim 33 comprising reacting octopamine, or one of its salts, in the presence of a base.

35. The process of claim 33 comprising reacting octopamine, or one of its salts, in the presence of a coupling reagent used in peptide synthesis.

36. The process of claim 30, wherein said carbonyl or sulfonyl derivative is an aldehyde, active or inactive ester, carboxylic or sulfonic acid chloride, anhydride or isocyanate.

37. The process of claim 33, wherein said carbonyl or sulfonyl derivative is an aldehyde, active or inactive ester, carboxylic or sulfonic acid chloride, anhydride or isocyanate.

38. A compound represented by one of the following formulas: 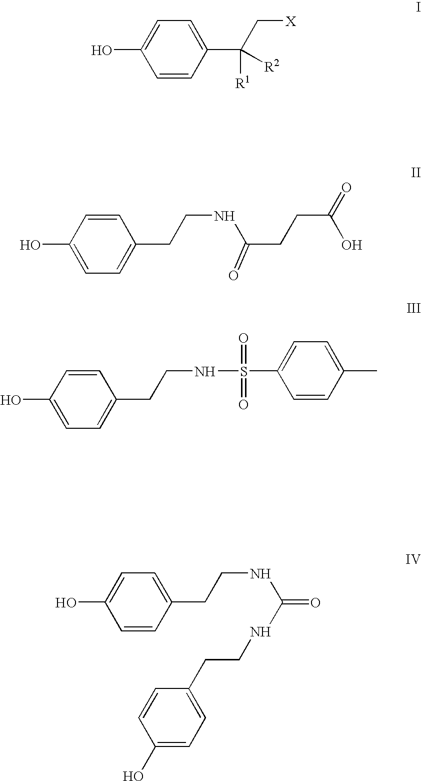
or a salt thereof, prepared by the process of claim 30 or 36.

39. The composition of claim 18, further comprising at least one material for the prevention or reduction of melanin production.

40. A compound comprising the following formula IV: 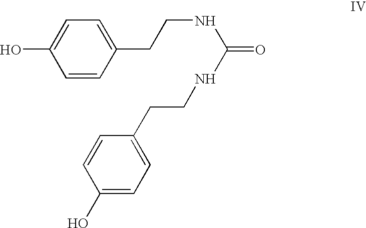
or a salt thereof.

41. An article comprising: the composition of claim 18 bound to, incorporated in, absorbed in or adsorbed on textiles, natural or synthetic fibers, wools, and any materials that may be used for clothing or for day or night underwear intended to come into contact with the skin or hair, to exert their cosmetic effect by means of skin or hair/textile contact and to permit continuous topical delivery.

42. A method for preventing or reducing melanogenesis comprising the step of administering to the skin, hair, mucosae, or a skin appendage the composition of claim 18.

43. A method for reducing coloration on exposure to natural or artificial UV radiation, lightening the complexion, attenuating senile lentigines, homogenizing the color of the skin or lightening any pigmentation associated with melanin comprising the step of administering to the skin, hair, mucosae, or a skin appendage the composition of claim 18.
